(19) United States
(12) Patent Application Publication
(10) Pub. No.: US 2019/0216295 A1
(43) Pub. Date: Jul. 18, 2019

(54) MEDICAL DEVICE

(71) Applicant: Olympus Corporation, Tokyo (JP)

(72) Inventors: Keisuke Hatano, Tokyo (JP); Tatsuhiko Suzuki, Tokyo (JP)

(73) Assignee: Olympus Corporation, Tokyo (JP)

(21) Appl. No.: 16/366,242

(22) Filed: Mar. 27, 2019

Related U.S. Application Data
(63) Continuation of application No. PCT/JP2017/037075, filed on Oct. 12, 2017.

(30) Foreign Application Priority Data
Nov. 17, 2016 (JP) 2016-224046

(51) Int. Cl. A61B 1/005; A61B 1/00; G02B 23/24

(52) U.S. Cl. CPC A61B 1/0052; A61B 1/00121; A61B 1/0057; A61B 1/00147; G02B 23/2476; A61B 1/00066

(57) ABSTRACT

The disclosed technology is directed to a medical device comprises an endoscope having an operation portion and a flexible insertion portion extending from the operation portion. The operation portion is used to manipulate the flexible insertion portion during an operation. The operation portion includes an elongated body defined by respective flat and incline surfaces having a predetermined angle with respect to one another on one side. A bending operation member mounted on the incline surface and projected outwardly therefrom toward the flat surface such that the bending operation member is protected by the flat surface so as to prevent the flexible insertion portion from unintentional bending when the endoscope not being in operation.

### CROSS-REFERENCE TO RELATED APPLICATIONS

This application is a continuation application of PCT Application No. PCT/JP2017/037075 filed on Oct. 12, 2017, which in turn claim priority to the Japanese Patent Application No. 2016-224046 filed on Nov. 17, 2016 in Japan which is hereby incorporated by reference in its entirety.

### TECHNICAL FIELD

The disclosed technology generally relates to a medical device and more specifically, to an endoscope having an operation portion at one end and an insertion portion at an opposed end for conducting examination into a subject. The operation portion includes an operation member that operates a bending portion disposed in the insertion portion.

### DESCRIPTION OF THE RELATED ART

In recent years, medical devices, for example, endoscopes have been widely utilized in the medical field.

It is well known that an endoscope is configured to include an insertion portion to be inserted into a subject, and an operation portion that is connectedly disposed on the proximal side of the insertion portion and is gasped by an operator.

The operation portion includes a bending operation member, and various operation switches and the like. The bending operation member is configured to bend an action portion or a bending portion disposed in the insertion portion.

International Publication WO 2016-147457 discloses a configuration of an endoscope wherein a joystick-type operation member is disposed as a bending operation member on an inclined surface having a predetermined angle relative to a longitudinal axis direction of an operation portion.

The joystick-type operation member includes a lever projected from the inclined surface, a finger resting portion which is disposed at a projecting end of the lever and on which an operator's finger is put, and a cover covering the outer periphery of the lever.

In the endoscope disclosed in International Publication WO 2016-147457, the operation portion is configured to put on a flat surface, such as a floor surface of a cleaning tank in a cleaning/disinfecting device for the endoscope or a floor surface of a sink or the like for cleaning the endoscope. In this case, the finger resting portion is directed to the side of the flat surface, and the finger mounting portion would make contact with the flat surface.

As a result, the lever is tilted down, so that the bending portion may bend unintentionally. Then the bending portion and a tip portion of the insertion portion may make contact with other body so as to be damaged.

If the action portion is other portion than the bending portion disposed in the insertion portion, the similar results happen. Also, the similar results happen in medical devices other than the endoscope.

The disclosed technology has been made in consideration of the circumstances described hereinbefore. Accordingly, it is an object of the disclosed technology to provide a medical device includes in which the action portion disposed in the insertion portion can be prevented from acting unintentionally by preventing the finger resting portion from making contact with a flat surface when an operation portion is mounted on the flat surface with the finger resting portion directed to the side of the flat surface.

### BRIEF SUMMARY OF EMBODIMENTS

A medical device according to an aspect of the disclosed technology includes an insertion portion that is inserted in a subject and has an action portion. An operation portion is connectedly disposed on a proximal side of the insertion portion and is grasped by an operator and can be mounted on a flat surface. An operation member causes the action portion to act in the operation portion. The operation member includes a lever projected from an inclined surface having a predetermined angle relative to a mounted surface of the operation portion mounted on the flat surface. A finger resting portion which is disposed at a projecting end of the lever and on which an operator's finger is mounted. The operation member is disposed at such a position relative to the inclined surface that when the mounted surface of the operation portion is mounted on the flat surface with the finger resting portion directed to the side of the flat surface and a gap is formed between the finger resting portion and the flat surface.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a front view depicting an external appearance of an endoscope according to a first embodiment.

FIG. 2 is a right side view depicting a mounted surface of an operation portion of the endoscope, in a state in which the mounted surface is mounted on a flat surface with a finger resting portion of an operation member directed to the side of the flat surface.

FIG. 3 is a view depicting a mounted surface of an operation portion of the endoscope according to a second embodiment, in a state in which the mounted surface is mounted on the flat surface with the finger resting portion of the operation member directed to the side of the flat surface.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

Embodiments of the disclosed technology will be described hereinafter, referring to the drawings. Note that the drawings are schematic ones, attention should be paid to the fact that the relations between thickness and width of each member, the ratios of thicknesses of the respective members and the like are different from the real ones, and, naturally, portions different in size relation or ratio are included among the drawings.

Note that in the embodiments described hereinafter, the endoscope will be described as an example of a medical device.

### Embodiment 1

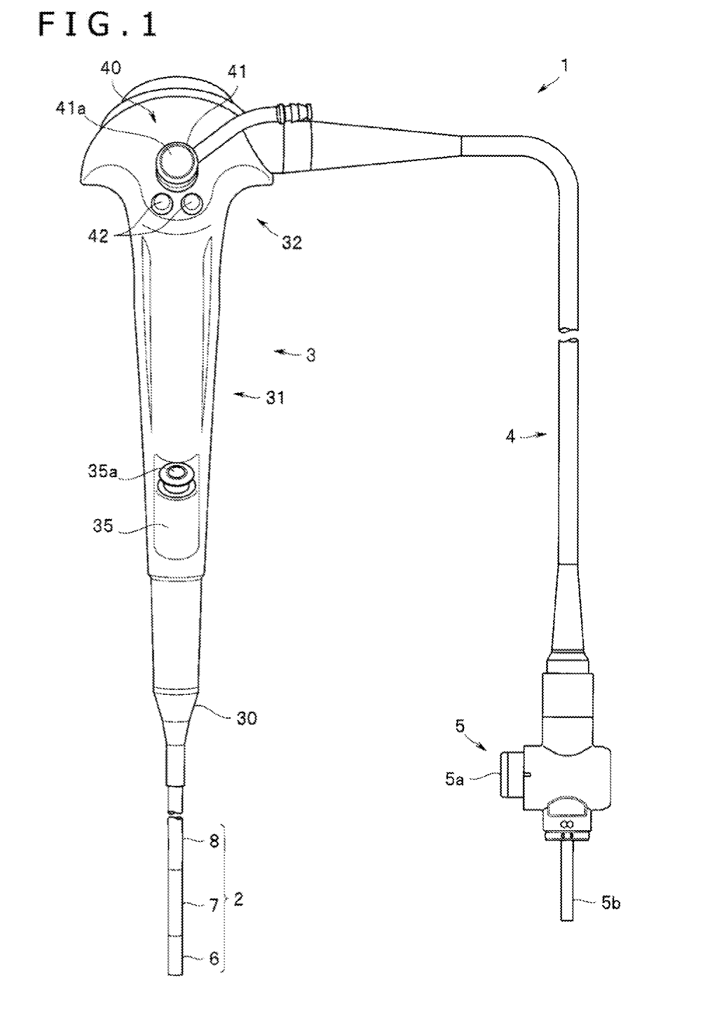

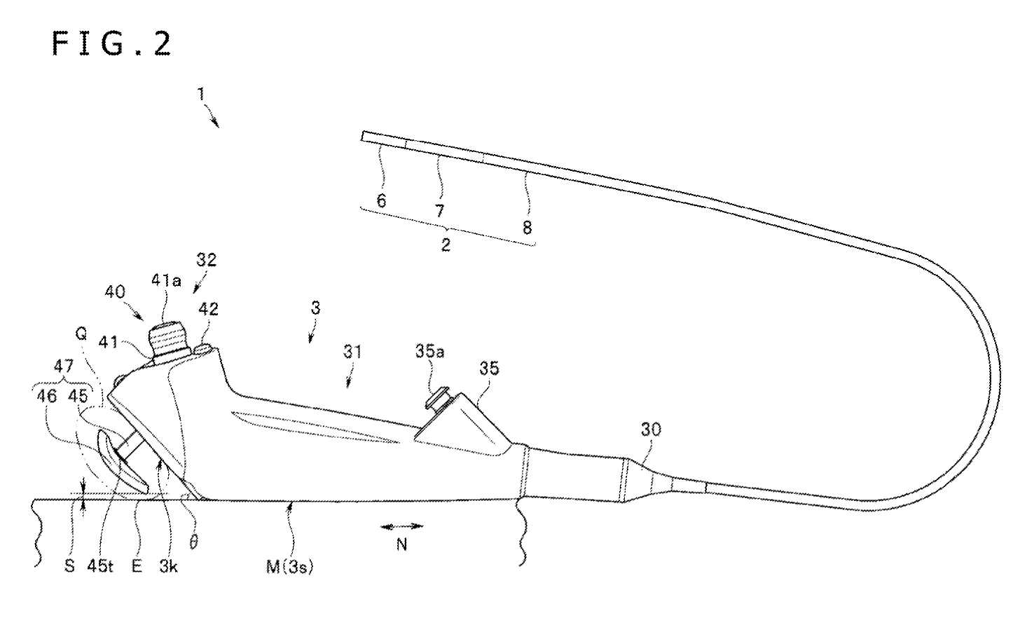

FIG. 1 is a front view depicting an external appearance of an endoscope of the present embodiment, and FIG. 2 is a right side view depicting a mounted surface of an operation portion of the endoscope of FIG. 1, in a state in which the mounted surface is mounted on a flat surface with a finger resting portion of an operation member directed to the flat surface.

As illustrated in FIGS. 1 and 2, the endoscope 1 includes an insertion portion 2 that is inserted in a subject, an operation portion 3 connectedly disposed on a proximal side of the insertion portion 2, a universal cord 4 extended from the operation portion 3, and an endoscope connector 5 disposed at an extension end of the universal cord 4.

The universal cord 4 is a composite cable in the inside of which various signal lines and the like extending from the side of a tip portion 6 through the inside of the insertion portion 2 to the operation portion 3 and further extending from the operation portion 3 are passed, a light guide (not depicted) is passed, and, further, an air-feeding and water-feeding tube (not depicted) is passed.

The endoscope connector 5 is disposed at a side surface portion thereof with an electrical connector portion 5a to which connects a signal cable connected to a video processor (not depicted), and is disposed at an end portion thereof with a light source connector portion 5b connected to a light source device (not depicted).

The insertion portion 2 includes the tip portion 6, a bending portion 7 used as an action portion, and a flexible pipe portion 8, in this order from the distal side, which constitute a main part.

The bending portion 7 is configured to be able to actively bend, for example, upward, downward, rightward and leftward directions (UP-DOWN/RIGHT-LEFT) according to an operation input given to the operation portion 3 by an operator.

The operation portion 3 is grasped by the operator, and can be mounted on a flat surface M. The operation portion 3 includes a boot 30 connected to the flexible pipe portion 8 in the state of covering a proximal end of the flexible pipe portion 8, a grasp portion 31 that is disposed connectedly to the boot 30 and is grasped by the operator's hand, and an operation portion main body 32 connectedly disposed on the proximal side of the grasp portion 31, which constitute a main part.

A treatment tool inserting and passing portion 35 is disposed on the front side in FIG. 1 on the distal side of the grasp portion 31. The treatment tool inserting and passing portion 35 has a treatment tool inserting and passing port 35a through which to insert a treatment tool (not depicted) into a treatment tool inserting and passing channel (not depicted) disposed in the insertion portion 2 and the operation portion 3.

Note that a forceps plug (not depicted) for closing the treatment tool inserting and passing port 35a can be attached to and detached from the treatment tool inserting and passing portion 35.

On the front side in FIG. 1 of the operation portion main body 32, there is disposed an operation button group 40 for performing various functions of the endoscope 1.

The operation button group 40 includes, for example, a suction button 41a projecting from a suction valve 41 which can be attached to and detached from the operation portion main body 32, and two button switches 42 to which arbitrary functions selected from among the various functions of the endoscope 1 can be assigned.

In addition, on the back side in FIG. 1 of the operation portion main body 32, there is disposed a mounted surface 3s that is mounted on the flat surface M and is substantially parallel to a longitudinal axis direction N of the operation portion 3, as depicted in FIG. 2.

Further, on the back side in FIG. 1 of the operation portion main body 32, there is disposed, continuously with the mounted surface 3s, an inclined surface 3k having a predetermined angle θ relative to the mounted surface 3s, as illustrated in FIG. 2.

Note that the inclined surface 3k may not be formed continuously with the mounted surface 3s, so long as the inclined surface 3k has the predetermined angle θ relative to the mounted surface 3s.

On the inclined surface 3k there is disposed a bending operation member 47 which is the operation member that causes the bending portion 7 to act, or to bend.

The bending operation member 47 is a so-called joystick-type operation member including a lever 45 projected from the inclined surface 3k and a finger resting portion 46 which is disposed at a projecting end 45t of the lever 45 and on which to position an operator's finger, and the lever 45 and the finger resting portion 46 constitute a main part.

Note that a watertight cover including a rubber or the like (not depicted) is covering the outer periphery of the lever 45. In addition, the projecting direction of the lever 45 may not necessarily perpendicular to the inclined surface 3k.

The bending operation member 47 is disposed at such a position relative to the inclined surface 3k that when the mounted surface 3s of the operation portion 3 is mounted on the flat surface M with the finger resting portion 46 directed to the side of the flat surface M, a gap S is formed between the finger resting portion 46 and the flat surface M.

Specifically, the finger resting portion 46 is located in a non-overlapping region Q in relation to an extension line E of the mounted surface 3s.

Note that as depicted in FIG. 2, when the mounted surface 3s is mounted on the flat surface M with the finger resting portion 46 directed to the side of the flat surface M, the operation portion 3 does not roll, or the state in which the mounted surface 3s is mounted on the flat surface M is maintained, because the universal cord 4 is extended from a right side surface in FIG. 1 of the operation portion main body 32.

Specifically, with the universal cord 4 making contact with the flat surface M, the state in which the mounted surface 3s is mounted on the flat surface M can be maintained.

Note that other points of configuration of the endoscope 1 are the same as in the related art.

Thus, in the present embodiment, it has been depicted that the bending operation member 47 is disposed at such a position relative to the inclined surface 3k that when the mounted surface 3s of the operation portion 3 is mounted on the flat surface M with the finger resting portion 46 directed to the side of the flat surface M, the gap S is formed between the finger resting portion 46 and the flat surface M.

Specifically, it has been depicted that the finger resting portion 46 is located in the non-overlapping region Q in relation to the extension line E of the mounted surface 3s.

According to this, when the mounted surface 3s of the operation portion 3 is mounted on the flat surface M with the finger resting portion 46 directed to the side of the flat surface M, the finger resting portion 46 would not contact the flat surface M.

Therefore, it is possible to securely prevent a situation in which the lever 45 brought into contact with the flat surface M might be tilted, the bending portion 7 might bend unintentionally, or the bending portion 7 or the tip portion 6 might come into contact with other body, to be thereby damaged.

From the foregoing, it is possible to provide the endoscope 1 having a configuration wherein an unintentional action of the bending portion 7 disposed in the insertion portion 2 can be prevented, by preventing the finger resting portion 46 from making contact with the flat surface M when the operation portion 3 is mounted on the flat surface M with the finger resting portion 46 directed to the side of the flat surface M.

### Embodiment 2

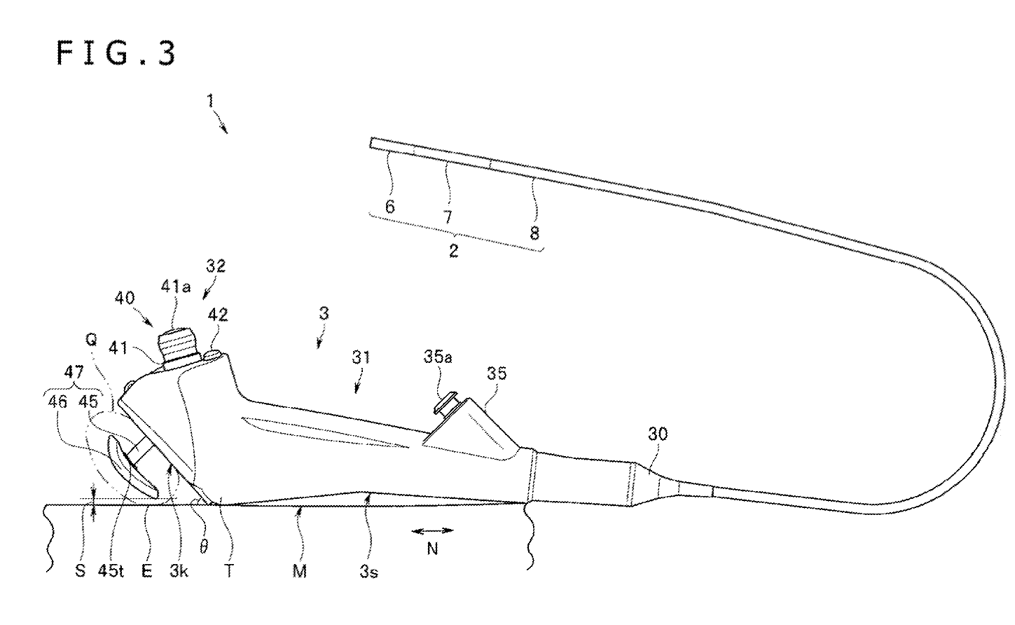

FIG. 3 is a figure depicting a state in which a mounted surface of an operation portion of an endoscope of the present embodiment is mounted on a flat surface with a finger resting portion of an operation member directed to the side of the flat surface.

The configuration of the endoscope of this second embodiment differs from that of the endoscope of the aforementioned first embodiment illustrated in FIGS. 1 and 2 in configuration in which a gap is formed between the finger resting portion and the flat surface when a mounted surface of the operation portion is mounted on the flat surface with the finger resting portion directed to the side of the flat surface.

Therefore, only this different point will be described, whereas components similar to those in the first embodiment are denoted by the same numeral reference as described hereinbefore, and descriptions of them will be omitted.

As illustrated in FIG. 3, in the present embodiment also, a bending operation member 47 is disposed at such a position relative to an inclined surface 3k that when the mounted surface 3s is mounted on the flat surface M with the finger resting portion 46 directed to the side of the flat surface M, the gap S is formed between the finger resting portion 46 and the flat surface M.

Specifically, the mounted surface 3s non-parallel to a longitudinal axis direction N is formed, in the vicinity of an inclined surface 3k, with a projecting portion T that projects from the mounted surface 3s and makes contact with the flat surface M, to thereby form the gap S. When the mounted surface 3s of the operation portion 3 is mounted on the flat surface M with the finger resting portion 46 directed to the side of the flat surface M, the gap S is formed between the finger resting portion 46 and the flat surface M due to the projecting portion T.

According to such a configuration, also, it is ensured that when the mounted surface 3s of the operation portion 3 is mounted on the flat surface M with the finger resting portion 46 directed to the side of the flat surface M, the finger resting portion 46 would not make contract with the flat surface M, so that an effect similar to that of the aforementioned first embodiment can be obtained.

Note that while the bending portion 7 has been depicted as an example of the action portion disposed in the insertion portion in the aforementioned first and second embodiments, this is not limitative, and other action portions may naturally be adopted.

In sum, one aspect of the disclosed technology is directed to a medical device comprises an endoscope having an operation portion and a flexible insertion portion extending from the operation portion. The operation portion is used to manipulate the flexible insertion portion during an operation. The operation portion includes an elongated body defined by respective flat and incline surfaces having a predetermined angle with respect to one another on one side. A bending operation member mounted on the incline surface and projected outwardly therefrom toward the flat surface such that the bending operation member is protected by the flat surface so as to prevent the flexible insertion portion from unintentional bending when the endoscope not being in operation.

The respective flat and incline surfaces are integrally attached to one another. The predetermined angle is defined by an angle that corresponds to a size of the bending operation member. The bending operation member and the flat surface are facing one another such that a gap is formed therebetween. The bending operation member is defined by a finger resting portion and a lever that are attached to one another. The bending operation member is attached to the inclined surface via the lever.

Another aspect of the disclosed technology is directed to a medical device comprises an endoscope having an operation portion and a flexible insertion portion extending from the operation portion. The operation portion is used to manipulate the flexible insertion portion during an operation. The operation portion includes an elongated body defined by respective flat and incline surfaces having a predetermined angle with respect to one another on one side. A bending operation member is mounted on the incline surface and projected outwardly therefrom toward the flat surface. The bending operation member is defined by a finger resting portion and a lever that are attached to one another such that the figure resting portion forms a gap with the flat surface and is protected by the flat surface so as to prevent the flexible insertion portion from unintentional bending when the endoscope not being in operation.

In addition, while the endoscope has been depicted as an example of the medical device in the aforementioned first and second embodiments, this is not restrictive, and other medical devices such as treatment tools may naturally be adopted.

Further, when the aforementioned first and second embodiments are applied to other medical devices having a gun-type grasp portion, also, a similar effect can be obtained.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. A medical device comprising:
an insertion portion configured to be inserted in a subject; and
an operation portion being connected on a proximal side of the insertion portion which being grasped by an operator, the operation portion having a surface configured to be disposed on a flat surface, the operation portion including an operation member having a lever projected from an inclined surface having a predetermined angle with respect to the flat surface, the operation member having a finger resting portion which is attached at a projecting end of the lever so as to permit the operator to manipulate,
wherein the operation member is configured to be disposed at such a position with respect to the flat surface so as to form a gap between the finger resting portion and the flat surface, and
wherein the surface of the operation portion includes a projecting portion configured to contact with the flat surface so as to form the gap.

2. The medical device according of claim 1,
wherein the surface of the operation portion is non-parallel to a longitudinal axis direction of the operation portion, and
the finger resting portion is located in a non-overlapping region in relation to an extension line of the flat surface.

3. The medical device according of claim 1,
wherein the insertion portion includes a bending portion disposed on a distal side of the insertion portion which inserted to the subject, and
the operation member is defined by a bending operation member causing the bending portion to bend.

4. The medical device according of claim 1, wherein the inclined surface is formed in continuation with the surface of the operation portion.

5. A medical device comprising:
an endoscope having an operation portion and a flexible insertion portion extending from the operation portion wherein the operation portion being used to manipulate the flexible insertion portion during an operation, the operation portion includes:
an elongated body defined by respective flat and incline surfaces having a predetermined angle with respect to one another on one side, a bending operation member mounted on the incline surface and projected outwardly therefrom toward the flat surface such that the bending operation member is protected by the flat surface so as to prevent the flexible insertion portion from unintentional bending when the endoscope not being in operation.

6. The medical device of claim 5, wherein the respective flat and incline surfaces are integrally attached to one another.

7. The medical device of claim 5, wherein the predetermined angle is defined by an angle that corresponds to a size of the bending operation member.

8. The medical device of claim 5, wherein the bending operation member and the flat surface are facing one another such that a gap is formed therebetween.

9. The medical device of claim 5, wherein the bending operation member is defined by a finger resting portion and a lever that are attached to one another.

10. The medical device of claim 9, wherein the bending operation member is attached to the inclined surface via the lever.

11. A medical device comprising:
an endoscope having an operation portion and a flexible insertion portion extending from the operation portion wherein the operation portion being used to manipulate the flexible insertion portion during an operation, the operation portion includes:
an elongated body defined by respective flat and incline surfaces having a predetermined angle with respect to one another on one side, a bending operation member mounted on the incline surface and projected outwardly therefrom toward the flat surface, the bending operation member is defined by a finger resting portion and a lever that are attached to one another such that the figure resting portion forms a gap with the flat surface and is protected by the flat surface so as to prevent the flexible insertion portion from unintentional bending when the endoscope not being in operation.
